(19) United States
(12) Patent Application Publication
(10) Pub. No.: US 2025/0384959 A1
(43) Pub. Date: Dec. 18, 2025

(54) SYSTEM AND METHOD FOR DESIGNING GRNA PROBES

(71) Applicant: MRIGlobal, Kansas City, MO (US)

(72) Inventors: Joseph Albert Russell, Frankford, DE (US); Phillip Evert Davis, Frederick, MD (US); Colin Webber Price, Falls Church, VA (US)

(21) Appl. No.: 19/199,020

(22) Filed: May 5, 2025

Related U.S. Application Data
(60) Provisional application No. 63/642,140, filed on May 3, 2024.

(51) Int. Cl. G16B 25/20; G16B 35/00

(52) U.S. Cl. CPC G16B 25/20; G16B 35/00

(57) ABSTRACT

This disclosure relates to systems, devices, and processes for designing gRNA sequences for use in CRISPR-based detection assays. In various embodiments, a process is provided including analyzing an input data set comprising one or more target inclusive sequences and a selected Cas protein, identifying one or more conserved k-mers within the data set, wherein the one or more conserved k-mers are substantially equal to a size required by the selected Cas protein, concatenating one or more scaffold sequences with the one or more identified conserved k-mers to create one or more candidate gRNA sequences, evaluating structural and specificity characteristics of the one or more candidate gRNA sequences, and displaying one or more output gRNA sequences, wherein the one or more output gRNA sequences are a subset created by removing any candidate gRNA sequences of the one or more candidate gRNA sequences not abiding to the structural and specificity requirements.

### CROSS-REFERENCE TO RELATED APPLICATIONS

This application claims the benefit of priority to U.S. Provisional Patent Application No. 63/642,140 filed May 3, 2024, the disclosure of which is incorporated herein by reference in its entirety.

### SUBMISSION OF SEQUENCE LISTING

The contents of the electronic submission of the Sequence Listing XML, titled 11202-234.xml, which was created on Sep. 8, 2025 and is 31,387 bytes in size, are incorporated herein by reference in its entirety.

### BACKGROUND

The adaptation of CRISPR technologies for molecular diagnostics marks a significant advancement in the field of biosurveillance and infectious disease response. CRISPR-based molecular detection systems leverage the specificity and versatility of CRISPR-Cas systems to detect genetic material. The functionality of these systems often hinges on the presence of a Protospacer Adjacent Motif (PAM) in the target DNA, which can be essential for the Cas enzyme to recognize and bind the target sequence. Compared to traditional PCR methods, CRISPR can prove an efficient alternative owing to specific advantages such as higher precision (due to reduced false signals and fast reaction time), elimination of non-specific amplification, and reduction of required genetic material, leading to faster time-to-results without the need for extensive amplification cycling. CRISPR-based detection systems can further offer superior specificity and sensitivity by directly binding and cleaving target DNA or RNA sequences, thus signaling the presence of specific pathogens.

However, like PCR, which requires primer and probe generation for unique genetic targets, CRISPR-based technology can require crafting unique bridging guide RNA (gRNA) sequences to interact with target DNA/RNA as well as the Cas protein. Thus, the efficacy of CRISPR technologies can heavily depend on the design of specific guide RNA (gRNA) sequences tailored for each genomic target, a process that can be intricate and time-consuming.

As a result, there is a long-felt, but unsolved need for platforms that allow for gRNA design and evaluation with a high degree of flexibility and modularity.

### BRIEF SUMMARY OF THE DISCLOSURE

In at least one embodiment, the present disclosure relates to a process for designing and evaluating specific gRNA probes for binding to a target DNA or RNA sequence. Advantageously, the disclosed processes can be scaled efficiently to problems of any input sequence size.

The following embodiments and aspects thereof are described and illustrated in conjunction with systems, devices, and processes that are meant to be exemplary and illustrative, not limiting in scope.

Briefly described, aspects of the present disclosure generally relate to CRISPR-Cas systems. According to a first aspect, the present disclosure relates to a process for designing gRNA sequences for use in CRISPR-based detection assays, the process comprising: analyzing an input data set comprising one or more target inclusive sequences and a selected Cas protein; identifying one or more conserved k-mers within the data set, wherein the one or more conserved k-mers are substantially equal to a size required by the selected Cas protein; concatenating one or more scaffold sequences with the one or more identified conserved k-mers to create one or more candidate gRNA sequences; evaluating structural and specificity characteristics of the one or more candidate gRNA sequences; and displaying one or more output gRNA sequences, wherein the one or more output gRNA sequences are a subset of the one or more candidate gRNA sequences, the subset created by removing any candidate gRNA sequences of the one or more candidate gRNA sequences not abiding to the structural and specificity requirements.

According to a second aspect, the process of the first aspect or any other aspect, further comprising retrieving one or more genome data sets associated with the one or more target inclusive sequences from a National Center for Biotechnology Information database.

According to a third aspect, the process of the first aspect or any other aspect, further comprising creating a genome data set using metadata associated with the genome data set.

According to a fourth aspect, the process of the first aspect or any other aspect, further comprising receiving requirements of the structural characteristics, the structural characteristics comprising at least one of a PAM sequence location, guanine/cytosine (GC) content, a scaffold sequence free energy, a gRNA free energy, and preservation of the scaffold sequence folding structure upon addition of the candidate gRNA sequence.

According to a fifth aspect, the process of the fourth aspect or any other aspect, further comprising identifying a PAM region within the one or more target inclusive sequences in a location required by the selected Cas protein.

According to a sixth aspect, the process of the fourth aspect or any other aspect, further comprising identifying a PAM region of a length required by the selected Cas protein within the one or more target inclusive sequences.

According to a seventh aspect, the process of the fourth aspect or any other aspect, wherein GC content of the candidate gRNA sequences is required to be between 40% and 60%.

According to an eighth aspect, the process of the first aspect or any other aspect, further comprising utilizing clustering or graphing operations to identify the one or more conserved k-mers among a set of k-mers selected for analysis

According to a ninth aspect, the process of the first aspect or any other aspect, wherein the selected Cas protein is Cas12.

According to a tenth aspect, the process of the first aspect or any other aspect, wherein the selected Cas protein is Cas13.

According to an eleventh aspect, the process of the first aspect or any other aspect, wherein an inclusive group and an exclusive group are defined using a BLAST database, wherein the inclusive group comprises a record of all genomes associated with the target inclusive sequences; and wherein the exclusive group comprises a record of one taxonomic tree-level above a taxonomy of the inclusive group.

According to a twelfth aspect, the process of the eleventh aspect or any other aspect, further comprising evaluating a specificity characteristic of inclusivity by determining matches between the one or more candidate gRNA sequences and the inclusive group and evaluating a specificity characteristic of exclusivity by determining matches between the candidate gRNA sequences and the exclusive group.

According to a thirteenth aspect, the process of the twelfth aspect or any other aspect, wherein at least one candidate gRNA sequence is at least 98% inclusive.

According to a fourteenth aspect, the process of the twelfth aspect or any other aspect, wherein at least one candidate gRNA sequence is at least 98% exclusive to taxonomic near neighbors.

According to a fifteenth aspect, the process of the eleventh aspect or any other aspect, further comprising evaluating the specificity characteristic of exclusivity to human signal by determining matches between the candidate gRNA sequences and the GRCh38 human genome.

According to a sixteenth aspect, the process of the fifteenth aspect or any other aspect, wherein at least one candidate gRNA sequence is at least 98% exclusive to the human genome.

According to a seventeenth aspect, the process of the first aspect or any other aspect, further comprising experimentally validating the output gRNA sequences via an experimental assay.

According to an eighteenth aspect, the present disclosure relates to a system for use in CRISPR-based detection assays, the system comprising: a computing device configured to receive at least one input data set comprising one or more target inclusive sequences and a selected Cas protein, the computing device comprising: a processor; and a memory device comprising a non-transitory storage medium encoded with instructions executable by the processor which, when executed by the processor, cause the processor to identify one or more conserved k-mers within the at least one input data set, wherein the one or more conserved k-mers are substantially equal to a size required by the selected Cas protein, concatenate one or more scaffold sequences with the one or more identified conserved k-mers to create one or more candidate gRNA sequences, evaluate structural and specificity characteristics of the one or more candidate gRNA sequences, and display one or more output gRNA sequences, wherein the one or more output gRNA sequences are a subset of the one or more candidate gRNA sequences, the subset created by removing any candidate gRNA sequences of the one or more candidate gRNA sequences not abiding to the structural and specificity requirements.

According to a nineteenth aspect, the system of the eighteenth aspect or any other aspect, wherein the instructions are coded in python script.

According to a twentieth aspect, the system of the eighteenth aspect or any other aspect, wherein the subset of output gRNA sequences is produced within 24 hours of receiving the input data set of target inclusive sequences and the selected Cas protein to the system.

According to a twenty-first aspect, the process of the eighth aspect or any other aspect, further comprising clustering the target inclusive sequences into one or more subclusters and using one or more k-mer hashes to identify one or more conserved k-mers from the one or more subclusters.

According to a twenty-second aspect, the process of the eighth aspect or any other aspect, further comprising representing the one or more target inclusive sequences as a hypergraph and calculating a minimum covering set to extract conserved k-mers from the target inclusive sequences.

The following discussion represents one embodiment of the systems and processes disclosed herein. It is to be understood that the following description should be considered non-limiting and is presented to enable a person skilled in the art to make and use embodiments of the disclosure. Various modifications to the illustrated embodiments are to be readily apparent to those skilled in the art, and the generic principles herein can be applied to other embodiments and applications without departing from embodiments of the disclosure. Thus, embodiments of the disclosure are not intended to be limited to embodiments shown but are to be accorded the widest scope consistent with the principles and features disclosed herein.

### BRIEF DESCRIPTION OF THE DRAWINGS

FIG. 1 is a flowchart depicting a process of designing a gRNA sequence for a CRISPR-Cas system, according to at least one embodiment;

FIG. 2 is a schematic representation of the inputs used and analyzed when following the process of FIG.1, according to at least one embodiment;

FIG. 3A is a schematic representation of folding behavior of an exemplary scaffold sequence (SEQ. ID. NOs. 31 and 32);

FIG. 3B is a schematic representation of folding behavior of the scaffold sequence of FIG. 3A after addition of a target sequence (SEQ. ID. NOs. 33 and 34); and

FIG. 4 is a schematic diagram depicting a process of validating a gRNA sequence produced following the process of FIG. 1 in a CRISPR study.

### DEFINITIONS

Before any embodiments are described in detail, it is to be understood that the disclosure is not limited in its application to the details of construction and the arrangement of components set forth in the following description or illustrated in the drawings, and is limited only by the claims that follow the present disclosure. The disclosure is capable of other embodiments, and of being practiced, or of being carried out, in various ways.

Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

According to various instances, this disclosure relates to systems and processes that improve the efficiency of designing gRNA probes for target inclusive sequences. The systems and processes herein are sometimes referred to as Cas-CRISPR Automated Design and Evaluation (“CasCADE”). In some cases, the CasCADE process can be provided in the form of an automated system to analyze input genomic reference sequences and identify sequences conserved across a plurality of records. CasCADE's rapid design process can render the tool critical in field-forward work, such as infectious disease surveillance, where quick results are essential.

As used herein, the terms k-mer, Cas protein, gRNA, and protospacer adjacent motif (PAM) region refer to terms of art as recognized by one of ordinary skill in the art in clustered regularly interspaced short palindromic repeats (“CRISPR”) technology. For instance, as used herein, the term “CRISPR-Cas system” refers to an enzyme system including a guide RNA sequence (e.g., sgRNA, crRNA) that contains a nucleotide sequence complementary or substantially complementary to a region of a target nucleic acid sequence, and a protein with nuclease activity.

CRISPR-Cas systems can include engineered and/or programmed nuclease systems derived from naturally occurring CRISPR-Cas systems. CRISPR-Cas systems can also include engineered and/or mutated Cas proteins. CRISPR-Cas systems can further contain engineered and/or programmed guide RNA. The terms “CRISPR-Cas system” and “CRISPR system” can be used interchangeably.

As used herein, a “k-mer” is a substring of a biological sequence, such as a nucleic acid sequence or a protein sequence, of length k that is extracted from a longer nucleic acid sequence (e.g., DNA, RNA) or protein sequence. In certain instances, a k-mer is used in bioinformatics for computational genomics and sequence analysis by breaking down larger sequences into more manageable segments.

As used herein, a Cas protein is an enzyme that cleaves target DNA or RNA strands for gene editing. In various instances, the Cas protein (also referred to as the “Cas endonuclease” or “Cas nuclease”) can comprise at least a tracrRNA region and a crRNA region. The tracrRNA region, in addition to one or more direct repeat regions comprising repeated oligonucleotide sections, can form a scaffold of the Cas protein. The crRNA region comprises a protospacer region intended for binding with the target DNA/RNA strand of the CRISPR-Cas system. In certain instances, the tracrRNA and the crRNA are linked via an end loop to form a guide RNA. In other instances, the CRISPR-Cas system does not feature a tracrRNA region and/or may include other structures which will be recognized by one of skill in the art.

As used herein, a PAM (Protospacer Adjacent Motif) region is a nucleotide sequence that interacts with a PAM-Interacting domain (PI domain) of the Cas protein. In various instances, the Cas protein initiates cleavage of the target DNA strand once the PI-domain has verified the PAM region. In certain instances, the PAM region is at least three nucleotides in length, but may be any suitable number of nucleotides in length.

As used herein, gRNA, or guide RNA, is an oligonucleotide RNA sequence that can or is designed to direct the cleavage activities of the CRISPR-Cas protein. In various instances, gRNA is comprised of a tracrRNA region and a crRNA region. In certain instances, a protospacer region of the crRNA is between 17 and 20 nucleotides in length. Without being bound to a particular theory, the gRNA is believed to guide the Cas protein to the appropriate cleavage site on the target DNA/RNA strand and determines the overall specificity of the CRISPR system to different target strands.

As used herein, a target inclusive sequence is a nucleotide sequence constituting the non-target strand of the target DNA/RNA that is not cleaved or is not to be cleaved. As will be understood herein, target inclusive sequences can be generally identical to gRNA of a CRISPR-Cas system owing to the complementarity of the target inclusive sequence and the gRNA with the target DNA/RNA strand. In some instances, a complete dataset of target inclusive sequences for a given Cas protein can be incongruent and feature substantial differences. In some such instances, the target inclusive sequences can feature similar sections in the form of sequences of a defined length (hereinafter referred to “conserved k-mers”).

### Detailed Description

According to particular embodiments, the present disclosure relates generally to a system and process for designing gRNA probes. More specifically, the present disclosure relates to designing gRNA that is highly inclusive, exclusive to taxonomic near neighbors, and exclusive to human signal when applied in a CRISPR-Cas system.

As will be understood herein, CRISPR-based molecular detection systems frequently utilize a guide RNA (gRNA) comprising a protospacer and a scaffold sequence that directs the Cas protein to bind specifically to the target DNA or RNA. Without being bound to a particular theory, accurate design of the gRNA is regarded as a crucial step in customizing a CRISPR-Cas protein system. In order to ensure precise and efficient gene modification, the gRNA in certain embodiments is designed with certain considerations in mind, including but not limited to the following:
A) On-Target Inclusivity: An ideally constructed gRNA is considered in the art to be inclusive (e.g., perfectly inclusive in some embodiments), wherein the gRNA binds to all intended target DNA/RNA sequences without error or delay. Thus, specified target inclusive sequences on the DNA/RNA target are considered “on-target” matches of the gRNA.
B) Off-Target Exclusivity: An ideally constructed gRNA is considered in the art to be exclusive (e.g., entirely exclusive in some embodiments) to all unintended target DNA/RNA sequences. Thus, any and all sequences incongruent with the specified target inclusive sequences on the DNA/RNA target, such as taxonomic near-neighbors (similar genetic material from closely related organisms or humans), are considered “off-target” matches of the gRNA. Off-target exclusivity can ensure high precision in identifying targets in such CRISPR-Cas systems.
C) Adherence to Structural Requirements: An ideally constructed gRNA is considered to follow all defined requirements of the target DNA/RNA, including but not limited to location of a PAM region, k-mer length, scaffold sequence details, and folding behavior.

The CasCADE process can thereby facilitate the production of specialized gRNA sequences intended for use in CRISPR applications, using the above criteria as goals of the output gRNA configurations. In various embodiments, the disclosed process can assess each potential candidate gRNA sequence's ability to detect specific genetic material while ensuring that the candidate gRNA sequence does not react with undesired sequences. Of course, although it is possible that not all suggested gRNA sequences provided by CasCADE exhibit the aforementioned “ideal” characteristics, such sequences can still be highly useful in the field of gene editing.

The CasCADE process can utilize a communication interface to request and receive any one or more of the disclosed items from an assay designer, including but not limited to selection of or a preference for a Cas protein, PAM sequence presence/location, GC content of the candidate gRNA sequences, scaffold sequence folding structure, etc. In some instances, the communication interface can be configured to interact with external programs, systems, interfaces, and the like to request and receive additional data relevant to gRNA design. In one or more instances, the communication interface can provide a graphical user interface (“GUI”) serving as an interaction point for the assay designer. For example, the communication interface can cause display of the GUI on a display screen (e.g., an LCD or LED display screen). The GUI can include various prompts, input boxes, etc. that can allow the communication interface to request and the assay designer to provide data from any one or more user devices. Through the GUI, assay designers can input requirements, preferences, and desired properties of the resulting CRISPR-Cas system.

The CasCADE interface can further be electronically coupled to a control device, including one or more processors and memory for storage and retrieval of processed information to be processed by the processor. The control device can operate autonomously or semi-autonomously and can read executable software instructions from the memory or a computer-readable medium (e.g., a hard drive, a CD-ROM, flash memory), or can receive instructions via an input from the assay designer, or another source logically connected to a computer or device, such as another networked computer or a server. For example, the server can be used to control the CasCADE interface via the controller on-site or remotely and the system may leverage one or more Application Programming Interfaces (APIs)

The memory can be provided in the form of a non-transitory storage medium encoded with instructions executable by the processor. In various instances, the instructions are coded using custom scripts, such as python.

Additionally, although the following discussion can describe features associated with specific devices or embodiments, it is to be understood that additional devices and/or features can be used with the described systems and processes, and that the discussed devices and features are used to provide examples of possible embodiments, without being limited.

### Parameters

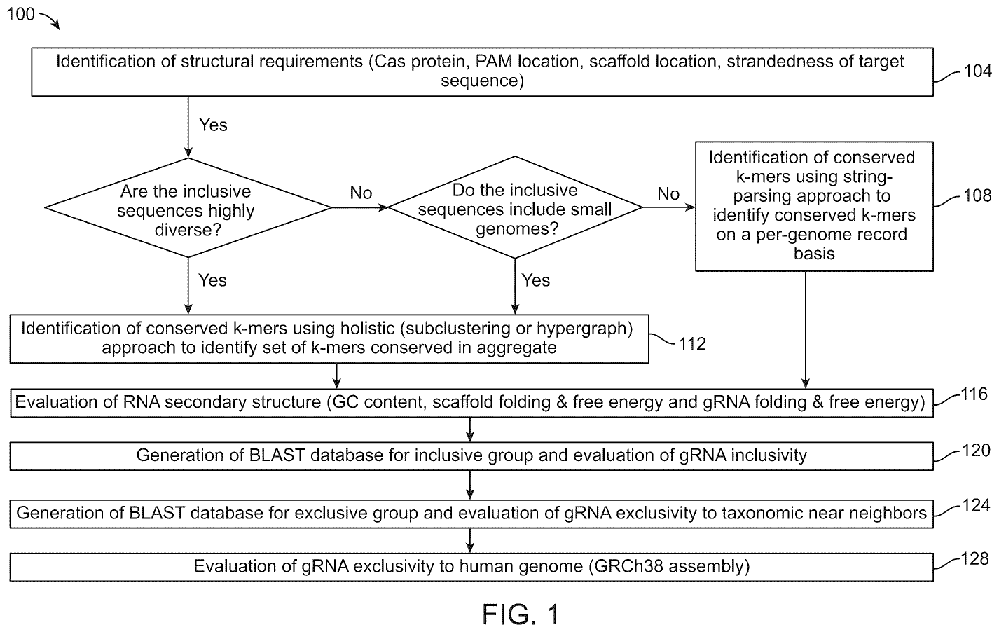

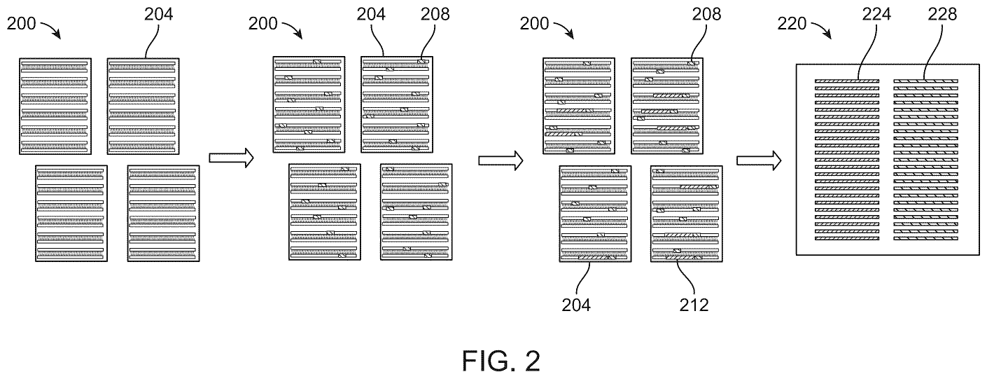

Referring now to FIGS. 1 and 2, the CasCADE process 100 can begin with step 104, which can include defining required structural parameters for the resulting CRISPR-Cas protein system.

Factors that can influence these design parameters for a given target include presence of PAM sites around a targeted region of interest, whether the target is DNA or RNA, desired temperature and ionic conditions at which the assay is to function, and a size of a Cas protein for delivery considerations. In at least one embodiment, the system may receive such structural parameters via a GUI (as discussed above) or from another computing system (e.g., via an API or the like).

In various instances, these structural parameters can be applied to a dataset, such as the dataset 200 displayed in FIG. 2, within a CasCADE interface. The dataset 200 can be comprised of one or more target inclusive sequences 204, indicative of sequences intended to be retained after CRISPR modification. The CasCADE process (system) 100 can automatically access the National Center for Biotechnology Information (NCBI) command line datasets tool in response to uploading of the target inclusive sequences 204. In some instances when the target inclusive sequences 204 are directly associated with a taxonomic label, the CasCADE process 100 can attempt to search for a genomic match between the inclusive sequences 204 and the genomes within the NCBI database and then download genomes associated with the target inclusive sequences 204 from NCBI upon locating a match.

In other instances the target inclusive sequences 204 do not correspond exactly to a set of one or more genomes available in the NCBI database, and the CasCADE process 100 can fail to find a match between the inclusive sequences 204 and a set of genomes within the NCBI database. In such instances, the assay designer can manually create a custom genome dataset within the CasCADE interface using, for example, a metadata labeling table. In some such instances, the assay designer can manually access the NCBI command line datasets tool to compile the custom genome dataset.

The Cas protein to be used as the primary nuclease enzyme in the CasCADE process 100 can in some instances be defined first as the one or more structural parameters input into the CasCADE interface in step 104. Stated differently, the system can receive the primary nuclease enzyme via the GUI or another mechanism (e.g., API). The number of known CRISPR-Cas systems has increased in recent years, and the classification of CRISPR-Cas systems is updated as new systems are discovered. CRISPR-Cas systems can be classified as Types I to VI based on the Cas protein in the system: For example, Cas9 can be found in Type II systems, and Cas12 can be found in Type V systems. Each Type can be further divided into subtypes. For example, Type II can include subtypes II-A, II-B, and II-C, and Type V can include subtypes V-A and V-B. A recent classification includes 2 classes, 6 types, and 33 subtypes.

The CRISPR-Cas systems and Cas nucleases described herein can encompass any Type or variant. Selection of the particular Cas protein to be used can be based on a variety of factors, including but not limited to AT/GC content of the target DNA/RNA, PAM compatibility, and known sequences in the target DNA/RNA. In certain non-limiting examples, the CRISPR-Cas12 protein is elected within the CasCADE interface. In certain other non-limiting examples, the CRISPR-Cas13 protein is elected within the CasCADE interface. In some such cases, the CRISPR-Cas system does not feature a tracrRNA region.

In some instances, existence of a PAM region in target DNA can be included as a required structural parameter in step 104 of the process 100. Without being bound to a particular theory, nucleotide sequence NGG is widely accepted as the canonical PAM region, although other minority sequences such as NGAG, NGN, NNGRRT, and others are also prevalent. Thus, the number, identity, and location of the PAM sequence of a target DNA can be specified within the CasCADE interface in accordance with one or more desired characteristics. In some instances, the number, identity, and location of the PAM sequence of a target DNA are specified in accordance with the requirements of the selected Cas protein. For example, in the case a Cas 12 protein is selected for the CRISPR-Cas system, the PAM sequence can be required to be present on the 5′ end of the target DNA.

In addition to or alternatively, order of the nucleotides within the PAM region can also be indicated in the CasCADE interface. For example, the PAM region can be required to contain one or more of the nucleotide bases A, T, C, and G, as well as one or more of representative placeholders R, Y, K, M, S, W, B, D, H, V, and N (shown here in IUPAC degenerate base symbols). One of ordinary skill in the art would thereby appreciate that the features of the PAM region required by the process 100 can be completely customized by the assay designer.

Further, in some such instances, a location of the PAM region can also be specified within the CasCADE interface. For example, in some cases, the PAM region can be required to be on the 5′ side of the target sequence. In other cases, the PAM region can be required to be on the 3′ side of the target sequence. In yet other cases, a location of the PAM region can be identified via a distance, such as three nucleotides, away from the target sequence. In such cases, it can be indicated that the PAM region is to be located at least one nucleotide, or at least two nucleotides, or at least three nucleotides, or at least four nucleotides, or at least five nucleotides, or at least six nucleotides, or at least seven nucleotides away from the target sequence, although it can be indicated that the PAM region be located a number of nucleotides away from the target sequence that is somewhat less or even greater than these values. In other such cases, it can be indicated that the PAM region be located exactly one nucleotide, or exactly two nucleotides, or exactly three nucleotides, or exactly four nucleotides, or exactly five nucleotides, or exactly six nucleotides, or exactly seven nucleotides away from the target sequence, although it can be indicated that the PAM region be located a number of nucleotides away from the target sequence that is somewhat less or even greater than these values.

As will be understood by one of ordinary skill in the art, any suitable combination of the PAM-related specifications can be used in the processes described herein without departing from the principles of this disclosure. For example, in certain instances, a PAM region can be required comprising a particular number of nucleotides in a specified order located on a particular side of and a specified distance away from the target sequence.

Thus, in the incoming dataset 200, the required PAM sites 208 can be identified after defining the Cas protein and before defining all other required structural parameters. In other instances, the required and/or preferred PAM sites 208 can alternatively be identified before defining the Cas protein. In yet other instances, the assay designer can choose to not require the presence of a PAM region in the target DNA. In an additional instance, the assay designer can require that a PAM region not be present in the target DNA.

It is to be understood that other parameters can be included within the CasCADE interface outside of the parameters explicitly discussed herein. In various instances, any one or more of these parameters can be indicated as either a requirement or a preference. By way of one non-limiting example, the assay designer using the CasCADE interface can choose to input only a preferred distance of the PAM sequence away from the target strand. The CasCADE interface can return potential PAM configurations both adhering to the one or more preferences and not adhering to the one or more preferences in, for example, an ordered list, to allow the assay designer to decide the importance of the preference in the final gRNA design.

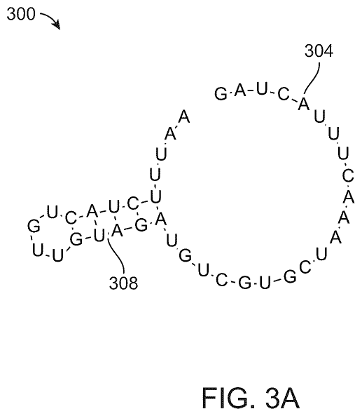

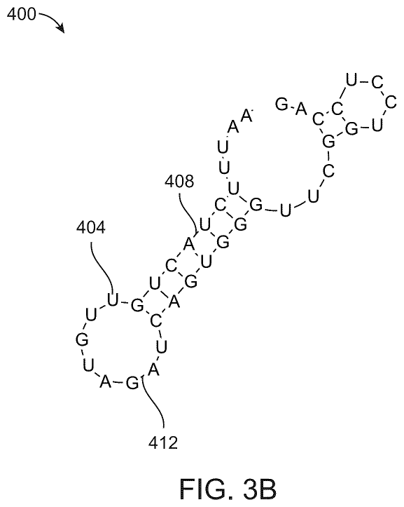

For example, in some instances, the assay designer can require that a scaffold sequence (see FIG. 3A) be located on the 5′ side or the 3′ side of the resulting gRNA designs. As another example, the assay designer can require that the target of the CRISPR-Cas system be limited to one or more of DNA and RNA. In some such cases, the DNA and/or RNA can be single-stranded, double-stranded, or both. Hence, the CasCADE interface is configured to allow for flexibility when selecting any of the previously described parameters for constraining the structure of the output gRNA designs.

The process 100 can then seek to identify a conserved k-mer 212 within the target inclusive sequences 204. A length of the conserved k-mer 212 can be specified prior to analysis of the dataset 200 for the conserved k-mer 212.

In various instances, the length of the conserved k-mer 212 is substantially equal to (such as, in some instances, no greater than 5% longer or shorter than) the length required by the chosen Cas protein. For example, in some instances, the conserved k-mer 212 can be specified as at least one nucleotide, or at least three nucleotides, or at least five nucleotides, or at least seven nucleotides, or at least ten nucleotides, or at least fifteen nucleotides, or at least twenty nucleotides in length, although it can be indicated that the conserved k-mer 212 is of a length that is less or even greater than these values. In other instances, the conserved k-mer 212 can be specified as exactly one nucleotide, or exactly three nucleotides, or exactly five nucleotides, or exactly seven nucleotides, or exactly ten nucleotides, or exactly fifteen nucleotides, or exactly twenty nucleotides in length, although it can be indicated that the conserved k-mer 212 is of a length that is less or even greater than these values.

It is to be understood by one skilled in the art that the conserved k-mer length required by a chosen Cas protein would be in various instances directly correlated to a length of the intended target DNA/RNA of the CRISPR-Cas system. Thus, in one example, it can be indicated that a particular target DNA or RNA strand of the system contains at least one nucleotide, or at least three nucleotides, or at least five nucleotides, or at least seven nucleotides, or at least ten nucleotides, or at least fifteen nucleotides, or at least twenty nucleotides, although it can be indicated that the particular target DNA or RNA strand is of a length that is somewhat less or even greater than these values. In other instances, it can be indicated that the particular target DNA or RNA strand contains exactly one nucleotide, or exactly three nucleotides, or exactly five nucleotides, or exactly seven nucleotides, or exactly ten nucleotides, or exactly fifteen nucleotides, or exactly twenty nucleotides, although it can be indicated that the particular target DNA or RNA strand is of a length that is somewhat less or even greater than these values.

In various instances, analysis of the dataset 200 for a conserved k-mer 212 is performed by the process 100 via a string-parsing approach 108. The process 100 can thereby analyze the target inclusive sequences 204 within the dataset 200 on a nucleotide-by-nucleotide basis and save instances where sets of identical nucleotides (of the preferred or required k-mer length specified by the assay designer) are located within each target inclusive sequence 204 of the dataset 200. In such instances, the target inclusive sequences 204 constituting the dataset 200 are likely of the same genome, and as such the conserved k-mer 212 can be evaluated by the process 100 on a per-genome record basis.

However, it will be appreciated by one of skill in the art that datasets 200 containing highly diverse target inclusive sequences 204 can feature conserved k-mers 212 that are not perfectly congruent. Similarly, target inclusive sequences 204 including particularly small genomes, such as a genome of an RNA virus, can present difficulty when seeking to identify exactly conserved k-mers over a wide dataset 200.

In some such instances, the process 100 can instead utilize a holistic approach 112 to identify a set of k-mers that are in aggregate conserved across the target inclusive sequences 204 as opposed to a singly conserved k-mer. Advantageously, the process 100 can be designed to automatically recognize when more than one genome is represented in the dataset 200 and switch operations to the holistic approach 112 when exactly conserved k-mers cannot be found. For example, the CasCADE process 100 can attempt to identify conserved k-mers 212 in all input inclusive sequences 104 using the string-parsing approach 108 and, upon determining that no (or too few) conserved k-mers are returned, automatically switch to the holistic approach 112. In other such instances, the assay designer can also enter manually that the holistic approach 112 is to be used for k-mer identification.

Following the holistic approach 112, the process 100 can in a first case employ a clustering operation, whereby the target inclusive sequences 204 are grouped into one or more subclusters. The grouping into subclusters can be performed using k-mer hashes, wherein a hash function executed by the CasCADE process 100 can organize the target inclusive sequences 204 constituting the dataset 200 for ordered searching of a completely conserved k-mer within each subcluster. Thus, as opposed to a singular perfectly conserved k-mer of the length required by the Cas protein being located for the dataset 200 as a whole, k-mers from each subcluster are identified. As a result, subclusters can be easily discriminated and identified in comparison to the time-consuming string parsing approach. In another case, the process 100 can instead employ a graph spanning operation. Herein, the CasCADE process 100 can use advanced algorithms to organize k'-mers in an unweighted hypergraph.

The hypergraph can feature the individual k-mers as nodes, with an indefinite number of edges connecting any two or more k-mers indicative of the membership of the k-mers within each target inclusive sequence 204. A minimum covering set can then be calculated for the hypergraph ensuring every target inclusive sequence 204 is represented in the output k-mer set. Once again, the plurality of output k-mers can be joined to form a resultant k-mer 212 conserved in portions throughout the dataset 200. Thus, advantageously, both operations may ensure high inclusivity of the dataset 200 while allowing for deviations within individual target inclusive sequences 204 as a result of genomic discrepancies.

In certain cases, the aforementioned operations can be performed internally by CasCADE programming and not require assistance or manipulation by the assay designer. In such cases, for example, the one or more subclusters and/or hypergraphs can be constructed solely as computational tools within the confines of the CasCADE programming. In other cases, these operations can be rendered graphically or artistically for visualization for the assay designer, such as on a GUI of the CasCADE interface. Thus, in such cases, the assay designer can have the option to, for example, modify the selected clustering scheme prior to analysis. In some such cases, the GUI can be configured to request confirmation or input from the assay designer at each computational “step”.

It is to be understood by one of ordinary skill in the art that additional specifications defined within the CasCADE process can also be used in the processes described herein without departing from the principles of this disclosure. In one non-limiting example, flanking sequences outside of the PAM region, can be evaluated as an additional preferred and/or required parameter within the CasCADE process 100. It is to be understood by one of skill in the art that a predefined Protospacer Flanking Sequence (PFS can represent nucleotide strings in the vicinity of a DNA region of interest-in many cases, the DNA target strand. In some such instances, the location of the flanking sequence on the 5′ or 3′ side of the target sequence as well as strandedness of the one or more flanking sequences, can be considered. In various instances, all of the Cas protein identity, PAM specifications, k-mer length, additional flanking sequences, and other considerations are indicated by the assay designer. In some other instances, one or more of the Cas protein identity, PAM specifications, k-mer length, additional flanking sequences, and other considerations are determined by the system based on other data input by an assay designer or received by the system.

Hence, the Cas-CRISPR Automated Design and Evaluation (CasCADE) platform as a pioneering bioinformatics tool can be designed to streamline the CRISPR assay setup and offers the advantage of uniquely facilitating the selection and optimization of essential parameters such as inclusion and exclusion groups, Cas protein types, scaffold sequences, Protospacer Adjacent Motif (PAM), and k-mer sizes as compared to traditional gRNA design processes.

### Structural Evaluation

Provided the inherent complementarity of both the conserved k-mer 212 and gRNA with the target DNA/RNA of the CRISPR-Cas system, it is to be understood that the spacer region of the candidate gRNA sequences designed by the CasCADE interface in various embodiments comprise, consist essentially of, or consist of the nucleic acid sequence of the conserved k-mer 212. Thus, in further steps of the CasCADE process 100, the CasCADE interface can treat the conserved k-mer 212 identified in steps 108, 112 as a preliminary form of a candidate gRNA sequence. The conserved k-mer 212 can be modified and evaluated via a series of structural assessments 116 to ensure viability of the conserved k-mer within the greater gRNA design.

In various instances, the conserved k-mer is modified such that thymine (T) bases are converted uracil (U) bases prior to any further modification or evaluation. In some such instances, the T-to-U conversion occurs automatically by the CasCADE process 100 without need for a user trigger.

Following identification of the conserved k-mer 212, the process 100 can thereby in various instances proceed in gRNA design with a step 116 by evaluating an expected folding pattern of a complete crRNA sequence (comprising the conserved k-mer 212) within the candidate gRNA sequence via a two-step process 116.

The process 100 can construct a first scaffold sequence to be later appended to the identified k-mer 212. In various instances, the scaffold sequence is representative of at least a portion of a tracrRNA region of the CRISPR-Cas system. The process 100 can first analyze the secondary structure and free energy of the scaffold sequence alone in conjunction with direct repeat regions to produce a first configuration, such as the structure 300 (SEQ. ID. NOs. 31 and 32) in FIG. 3A. The structure 300 can feature constituent bases 304 both paired and unpaired in various patterns.

The scaffold sequence can then be joined with the k-mer 212 to form a complete candidate gRNA sequence. In some instances, the process 100 can elect an end of the k-mer 212 (5′ end or 3′ end) at which to attach the scaffold sequence based on the properties of the selected Cas protein. In other instances, the assay designer can decide and indicate the appropriate end at which to join the scaffold sequence and k-mer 212, such as based on the location of the PAM sequence. In yet other instances, the process 100 can have an “override” feature such that an automatic selection of the end of the k-mer can be changed upon input by the assay designer. It is contemplated that such an override feature can as well be included for all other components of the process 100, wherein an automated decision can be altered provided the assay designer's particular desired inputs.

The process 100 can then determine an effect of the addition of the target sequence on the structure 300 by evaluating the folding behavior of the candidate gRNA sequence. It is contemplated that in some instances, the direct repeat plus scaffold sequence structure 300 can be disrupted by the presence of the target sequence. For example, in the case of the structure 400 (SEQ. ID. NOs. 33 and 34) in FIG. 3B, paired bases 408 are changed from the paired structures 308 featured in FIG. 3A. Herein, addition of the target sequence can thus lead to reshaping and non-conservation of the scaffold structure. Such instances where the crRNA structure is substantially changed as a result of the target sequence can be disregarded/deprioritized when considering a candidate gRNA sequence for later implementation.

In some instances, the RNA secondary structure of selected cRNA sequences can be visualized in SVG format.

The process 100 can consequently construct additional scaffold sequences for attachment to the identified k-mer 212 to compare properties of multiple candidate gRNA sequences. In various instances, other potentially useful metrics of the scaffold sequence include one or more of GC (guanine/cytosine) content and gRNA free energy compared to the scaffold sequence free energy.

These data can be tabularized or compiled into lists 224 and 228 for direct comparison. In some instances, the candidate gRNA sequences can be filtered down to a subset of top candidates by applying a weight or priority to certain parameters. In one non-limiting example, the assay designer can elect that the optimal candidate gRNA sequence presents the most stable (i.e., lowest) free energy value within a specified GC content range. In another example, the assay designer can instead elect that the optimal candidate gRNA sequence contains a preserved scaffold sequence structure. Similar to the procedure for PAM region specification, the assay designer can retain complete customization capabilities when selecting the attributes that provide for the most desired candidate gRNA sequence, as well as the requirement and/or preference for selected attributes. In some instances, the process can output only the candidate gRNA sequences abiding by the indicated structural requirements as a subset of the group of candidate gRNA sequences.

For example, the required GC content can be indicated as between about 40 and about 60 percent. In some instances, the required GC content can be indicated as between about 1 and about 90 percent, or between about 10 and about 80 percent, or between about 20 and about 80 percent, or between about 25 and about 75 percent, or between about 30 and about 70 percent, or between about 35 and about 65 percent. In other instances, the required GC content can be indicated as at least about 1 percent, or at least about 5 percent, or at least about 10 percent, or at least about 15 percent, or at least about 20 percent, or at least about 25 percent, or at least about 30 percent, or at least about 40 percent, or at least about 50 percent. In various embodiments, it can be indicated that the required GC content is in a range bound by any of these values, or in a range bound by percentages somewhat less or even greater than these values.

As another example, the required GC content can be indicated as between 40 and 60 percent. In some instances, the required GC content can be indicated as between 1 and 90 percent, or between 10 and 80 percent, or between 20 and 80 percent, or between 25 and 75 percent, or between 30 and 70 percent, or between 35 and 65 percent. In other instances, the required GC content can be indicated as at least 1 percent, or at least 5 percent, or at least 10 percent, or at least 15 percent, or at least 20 percent, or at least 25 percent, or at least 30 percent, or at least 40 percent, or at least 50 percent. In various embodiments, it can be indicated that the required GC content is in a range bound by any of these values, or in a range bound by percentages somewhat less or even greater than these values.

In certain cases, an output of the structural evaluation 116 can be provided by the CasCADE process 100 in BED format comprising a tabular output for reporting the aforementioned metrics. For example, the output tables can exhibit the numerical quantities for features such as the scaffold sequence and gRNA free energy while presenting Boolean results (e.g., True/False) for parameters such as structure preservation. The process 100 can further be configured to compare these metrics against saved gRNA profiles for further analysis of particular areas of interest within the gRNA design. Usage of the BED format herein can further allow for downstream genome alignment visualization by placing genomes side-by-side for facile comparison. Output in BED format can also assist in down-selection of specific gene targets of interest by comparing genome feature format files.

### Specificity Evaluation

Upon acquisition of a subset of candidate gRNA sequences that include desired properties, the process 100 can subsequently attach a label to the resulting tabular data file 220, as displayed in FIG. 2. The label can highlight the target inclusive sequences 204 used in the generation of the subset and can contain information such as taxonomic identification (or “tax ID”), referring to the one or more genomes associated with the target inclusive sequences 204. Thus, in some instances (such as those where genomic information was directly imported from the NCBI database), the label can be selected from predetermined labels stored in the CasCADE interface or, in other instances, the label can be customized by the assay designer to represent the unique combination of genomes represented in the dataset 200.

The process 100 can in some instances evaluate inclusivity of the subset of candidate gRNA sequences by comparing each candidate gRNA sequence to an entire inclusive group. In some such instances, the inclusive group can be comprised of, consist essentially of, or consist of the set of all records belonging to the target inclusive sequences 204 (in other words, all genomes represented in the tax ID) via a Basic Local Alignment Search Tool (BLAST) procedure 120. In certain cases, the inclusive record(s) can be automatically retrieved from NCBI datasets. The BLAST procedure 120 can primarily create a BLAST database comprising the entire inclusive group and search for alignments between the candidate gRNA sequences and the inclusive group. Matches located between the two datasets can be compiled into a first “hit record”. It is to be understood by one skilled in the art that an objective of perfect inclusivity would yield a BLAST result of no more than a single mismatched nucleotide, an exact length match of the k-mer 212 within the candidate gRNA sequences and the intended target sequences within the inclusive group, and an inclusivity of 100%. In some cases, the process 100 can flag or alert the assay designer of any inconsistencies beyond the single mismatched nucleotide. In other cases, the process 100 can notify the assay designer of mismatches only once exceeding a predefined threshold (for example, four mismatches between the candidate gRNA sequences and the inclusive group). In various instances, the process 100 can output a tabular report of the percent inclusivity, wherein the inclusivity is preferably 100%, as well as the total number of hits in the hit record. In particular instances, the candidate gRNA sequences can viably hit a genomic record more than one time.

In some cases, the inclusivity of the candidate gRNA sequences is at least about 95%. For example, in some such cases, the inclusivity of the candidate gRNA sequences is at least about 95%, or at least about 96%, or at least about 97%, or at least about 98%, or at least about 99%. In other such cases, the inclusivity of the candidate gRNA sequences is at least 95%, or at least 96%, or at least 97%, or at least 98%, or at least 99%.

In some instances, the inclusivity evaluation step 120 can further include parsing to ensure a desired location of the PAM region.

The process 100 can in some instances additionally evaluate exclusivity of the subset of candidate gRNA sequences by comparing each candidate gRNA sequence to an entire exclusive group. In some such instances, the exclusive group can be comprised of, consist essentially of, or consist of all records in one taxonomic tree-level record above the tax ID. By way of one non-limiting example, an inclusive group constituting one specie can be paired with an exclusive group constituting the overarching genus. In certain cases, the exclusive group can be automatically retrieved from NCBI datasets. A BLAST procedure 124 can primarily create a BLAST database comprising the exclusive group and search for alignments between the candidate gRNA sequences and the exclusive group. Matches located between the two datasets are compiled into a second “hit record”. It is to be understood by one of skill in the art that an objective of perfect exclusivity will yield a BLAST result of at least two mismatched nucleotides and exclusively hits to any target inclusive sequences 204 within the exclusive group. “Off-target” hits can thereby be defined as hits occurring to genomes within the exclusive group not represented in the inclusive group. In some cases, the process 100 can flag or alert the assay designer of any off-target hits. In other cases, the process 100 can notify the assay designer of off-target hits only once exceeding a predefined threshold (for example, four off-target hits). In various instances, the process 100 can output a tabular report of the percent exclusivity, wherein the exclusivity is preferably 100%, as well as the total number of hits in the hit record. In some instances, the report can comprise at least two tables, one providing the off-target hits in terms of accession number (i.e., a sequence identifier from the NCBI database), and a second table providing the off-target hits in terms of the taxonomic association (for example, a name of the genus where the off-target hit was found). In other instances, a greater or fewer number of tables can be provided in the output report indicating the results of the exclusivity analysis.

In some instances, a hit to a genome in the exclusive group is not considered an off-target hit when a specified PAM region does not occur at the required 5′ or 3′ side. In such instances, the PAM region is not located in the region of interest and therefore does not indicate a true “match” between the candidate gRNA sequence and the genome. In some such instances, the assay designer can elect that such non-off-target hits resulting from incorrectly placed PAM regions be tabularized or otherwise marked by the CasCADE interface.

The exclusivity determination step 124 can in some cases be repeated against the NCBI's database of genomic nucleotides if multiple rounds of testing are desired. For example, the assay designer can elect to examine a broader exclusivity group by evaluating the taxonomic record two tree-levels above the tax ID, or three tree-levels above the tax ID, or four tree-levels above the tax ID, and so on.

Further, in some additional cases, the process 100 can perform an additional exclusivity analysis 128 to determine whether one or more of any off-target hits by the candidate gRNA sequence and PAM sequence correspond to the human genome, also referred to as the GRCh38 assembly. In some such cases, an off-target human hit is outputted as a Boolean “True” by the process 100 to indicate non-exclusivity to the assay designer.

In some cases, the exclusivity of the candidate gRNA sequences to any exclusive group (such as one taxonomic tree-level above the tax ID or the human genome) is at least about 95%. For example, in some such cases, the exclusivity of the candidate gRNA sequences is at least about 95%, or at least about 96%, or at least about 97%, or at least about 98%, or at least about 99%. In other such cases, the exclusivity of the candidate gRNA sequences is at least 95%, or at least 96%, or at least 97%, or at least 98%, or at least 99%.

Advantageously, the described systems and processes can be designed to produce a subset of candidate gRNA sequences comprising, consisting essentially of, or consisting of candidate gRNA sequences that are highly inclusive, exclusive to taxonomic near neighbors, and exclusive to human signal, which one of skill in the art would appreciate as the ideal candidate for use in a detection assay.

The subset of candidate gRNA sequences subject to specificity evaluation can comprise any appropriate number of candidate gRNA sequence designs. For example, in some instances, the subset can include at least one candidate gRNA sequence, or at least two candidate gRNA sequences, or at least five candidate gRNA sequences, or at least ten candidate gRNA sequences, or at least fifteen candidate gRNA sequences, or at least twenty candidate gRNA sequences, or at least thirty candidate gRNA sequences, or at least fifty candidate gRNA sequences, or at least one hundred candidate gRNA sequences. The number of candidate gRNA sequences can be elected by the assay designer in accordance with parameters such as computational cost, tractability, and efficiency. For example, the assay designer can in some instances desire a complete assay design period of no more than about 24 hours. Therefore, on occasions where more than 24 candidate gRNA sequences pass the aforementioned filtration steps and are subject for specificity evaluation, the process 100 can instead randomly sample the more than 24 candidate gRNA sequences to produce a subset of 24 candidates to evaluate for inclusivity and exclusivity. In some such occasions, the assay designer can instead elect to produce a subset of fewer than 24 candidates to evaluate for exclusivity. In other cases, the assay designer can manually select the subset of 24 candidates to be evaluated.

The innovations discussed herein with respect to the CasCADE process 100 thereby improve at least the identification of conserved sequences across selected genomic datasets and offers the advantage of conducting enhanced assessment of critical properties such as structural integrity, energy stability, and GC content in comparison with existing/previous systems and processes.

### EXAMPLES

A demonstration of the viability of the gRNA designs produced following the CasCADE process 100 is provided in Table 1, denoting 15 experimentally validated gRNA designs for nine distinct targets, as well as the number of relevant target inclusive sequences used in designing (i.e., records), the guide sequence, and the guide plus the scaffold sequence.

TABLE 1

| Target | Records | Guide | Scaffold + Guide |
|---|---|---|---|
| Abrin Toxin | 14 | CACUGAACGUAUAGACC | UAAUUUCUACUAAGUGUAGAU |
| | | CAU | CACUGAACGUAUAGACCCAU |
| | | (SEQ. ID. NO. 1) | (SEQ. ID. NO. 16) |
| B. mallei & | 68 | UGAUAGCUUGAGAUCCC | UAAUUUCUACUAAGUGUAGAU |
| psuedomallei | | AUGCACUGAACGUAUAG | UGAUAGCUUGAGAUCCCAUG |
| | | ACCCAU | (SEQ. ID. NO. 17) |
| | | (SEQ. ID. NO. 2) | |
| B. mallei & | 68 | GUCUCUAUGGAAUCACU | UAAUUUCUACUAAGUGUAGAU |
| psuedomallei | | GCG | GUCUCUAUGGAAUCACUGCG |
| | | (SEQ. ID. NO. 3) | (SEQ. ID. NO. 18) |
| B. mallei & | 68 | AUGGCCAAUCCUGCAGA | UAAUUUCUACUAAGUGUAGAU |
| psuedomallei | | AUC | AUGGCCAAUCCUGCAGAAUC |
| | | (SEQ. ID. NO. 4) | (SEQ. ID. NO. 19) |
| Pan-Candida | 16 | UGUUCUCCAUGAGUCCC | UAAUUUCUACUAAGUGUAGAU |
| genus | | CCU | UGUUCUCCAUGAGUCCCCCU |
| | | (SEQ. ID. NO. 5) | (SEQ. ID. NO. 20) |
| Pan-Candida | 16 | ACUUCACGUUCGGUUCA | UAAUUUCUACUAAGUGUAGAU |
| Genus | | UCC | ACUUCACGUUCGGUUCAUCC |
| | | (SEQ. ID. NO. 6) | (SEQ. ID. NO. 21) |
| K. aerogenes | 99 | UAGAGAAUGACAGACGG | UAAUUUCUACUAAGUGUAGAU |
| | | CUU | UAGAGAAUGACAGACGGCUU |
| | | (SEQ. ID. NO. 7) | (SEQ. ID. NO. 22) |
| K. aerogenes | 99 | CAUUUUCAAAGCCGCCG | UAAUUUCUACUAAGUGUAGAU |
| | | GAA | CAUUUUCAAAGCCGCCGGAA |
| | | (SEQ. ID. NO. 8) | (SEQ. ID. NO. 23) |
| K. aerogenes | 99 | GCCAGACCGAUGUUAAA | UAAUUUCUACUAAGUGUAGAU |
| | | CCU | GCCAGACCGAUGUUAAACCU |
| | | (SEQ. ID. NO. 9) | (SEQ. ID. NO. 24) |
| K. | 529 | AUCGCAUCAAACUCUUC | UAAUUUCUACUAAGUGUAGAU |
| pneumoniae | | GGU | AUCGCAUCAAACUCUUCGGU |
| | | (SEQ. ID. NO. 10) | (SEQ. ID. NO. 25) |
| K. | 529 | AUCAAUUGGUCGCAGGU | UAAUUUCUACUAAGUGUAGAU |
| pneumoniae | | UCG | AUCAAUUGGUCGCAGGUUCG |
| | | (SEQ. ID. NO. 11) | (SEQ. ID. NO. 26) |
| P. falciparum | 18 | GAUACUUCGAACGCAUG | UAAUUUCUACUAAGUGUAGAU |
| | | ACC | GAUACUUCGAACGCAUGACC |
| | | (SEQ. ID. NO. 12) | (SEQ. ID. NO. 27) |
| P. aeruginosa | 1583 | UCUAUCGCGGUGAUUUG | UAAUUUCUACUAAGUGUAGAU |
| | | UCG | UCUAUCGCGGUGAUUUGUCG |
| | | (SEQ. ID. NO. 13) | (SEQ. ID. NO. 28) |
| Ricin | 51 | GACCUACGAUACGCACU | UAAUUUCUACUAAGUGUAGAU |
| | | AUG | GACCUACGAUACGCACUAUG |
| | | (SEQ. ID. NO. 14) | (SEQ. ID. NO. 29) |
| V. vulnificus | 54 | UUUUGGAGCUGUUUACU | UAAUUUCUACUAAGUGUAGAU |
| | | GGC | UUUUGGAGCUGUUUACUGGC |
| | | (SEQ. ID. NO. 15) | (SEQ. ID. NO. 30) |

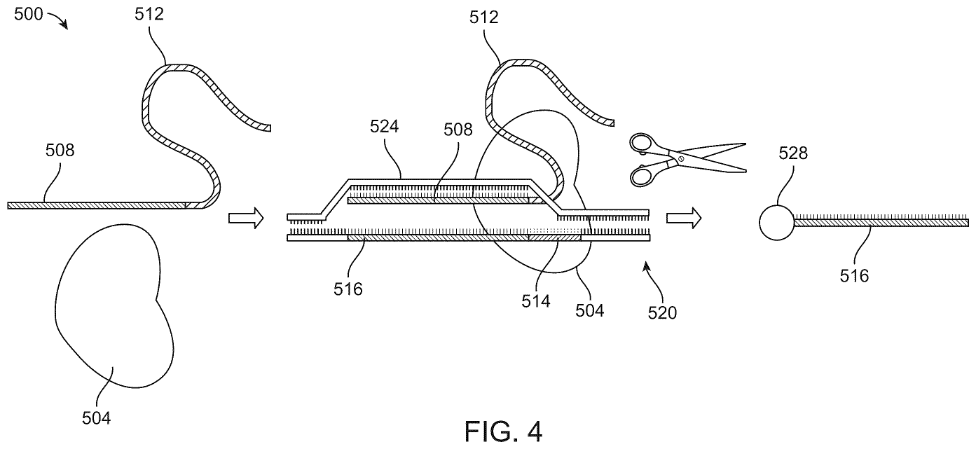

The designed assays were verified via use in a CRISPR study 500, as shown in FIG. 4. A CRISPR-Cas protein 504 was selected for use with the manufactured gRNA sequences 508 attached to scaffold sequences 512. After verification of a PAM region 514 in a non-target strand 516 of a target DNA 520, active gRNA of the Cas protein 512 binds to a target strand 524 of the target DNA 520. Once bound, the Cas protein 504 can cleave the region 524. In the study 500, the reporter mechanism 528 acts upon the freed non-target strand 516 and induces fluorescence, whose signal can be detected visually and/or quantitatively.

Thus, a gRNA design 508 is considered to successfully detect the target strand 524 if fluorescence is detected in the replicate 516. When performed, success was measured when detected fluorescence was greater than a background (or “negative”) fluorescence value plus three standard deviations. All 15 gRNA designs above successfully demonstrated a relative fluorescence unit (RFU) 1.3 to 2.3 times greater than the background control. Results are detailed in Table 2, displaying the above-referenced gRNA designs along with the limit of detection (LOD) of fluorescence, corresponding fluorescence signal of the target in RFU, background (BG) fluorescence signal in RFU, a difference between the target and background signals in RFU, and a ratio of the target to background fluorescence signal.

TABLE 2

| | | | | | Target − | |
|---|---|---|---|---|---|---|
| # | Target | LOD | Target (RFU) | BG (RFU) | BG (RFU) | Target:BG |
| 1 | Abrin Toxin | 50000 | 24955 | 11952 | 13003 | 2.1 |
| 2 | B. mallei & | 1000000 | 19667 | 12296 | 7371 | 1.6 |
| | psuedomallei | | | | | |
| 3 | B. mallei & | 100000 | 30396 | 17742 | 12654 | 1.7 |
| | psuedomallei | | | | | |
| 4 | B. mallei & | 1000000 | 18358 | 13748 | 4610 | 1.3 |
| | psuedomallei | | | | | |
| 5 | Pan-Candida | 1000000 | 48432 | 21102 | 27330 | 2.3 |
| | genus | | | | | |
| 6 | Pan-Candida | 1000000 | 23274 | 13349 | 9925 | 1.7 |
| | Genus | | | | | |
| 7 | K. aerogenes | 1000000 | 32216 | 17189 | 15027 | 1.9 |
| 8 | K. aerogenes | 1000000 | 25365 | 13929 | 11436 | 1.8 |
| 9 | K. aerogenes | 1000000 | 22802 | 13954 | 8848 | 1.6 |
| 10 | K. | 1000000 | 28254 | 21916 | 6338 | 1.3 |
| | pneumoniae | | | | | |
| 11 | K. | 1000000 | 42958 | 22349 | 20609 | 1.9 |
| | pneumoniae | | | | | |
| 12 | P. falciparum | 1000000 | 19775 | 13178 | 6597 | 1.5 |
| 13 | P. aeruginosa | 500000 | 32114 | 20908 | 11206 | 1.5 |
| 14 | Ricin | 350000 | 40504 | 20041 | 20463 | 2 |
| 15 | V. vulnificus | 1000000 | 30324 | 17625 | 12699 | 1.7 |

Advantageously, the results displayed in Table 2 exemplify how the deployment of the CasCADE platform can represent a significant advancement in the design of guide RNA for diverse biological targets, including viruses, fungi, bacteria, plants, and animals, underpinning broad-spectrum pathogen detection capabilities. In various instances, gRNA sequences designed using the CasCADE platform can be further validated in vitro, and such validation steps can be implemented in training procedures to refine the scoring and selection processes embedded in the platform to achieve enhanced efficiency. In particular instances, the validation and refinement steps can be performed iteratively to optimize the technical parameters required by the platform and broaden the subset of candidate gRNA sequences that can be produced for precise, reliable applicability in molecular diagnostic needs with even the most stringent requirements.

In some such instances, accumulation of data by the platform can allow for training steps to be provided to the program in the form of machine learning techniques. These techniques can leverage the vast array of data generated by CasCADE and allow for greater predictability by the program based on empirical outcomes. In various embodiments, modularity of the CasCADE pipeline can allow for the seamless integration of new design criteria or adjustments to existing parameters, ensuring that the platform remains adaptable to emerging scientific insights and diagnostic needs while providing comprehensive coverage and inclusivity of the target spectra. Machine learning techniques can further be developed to build models intended for prediction of in silico (i.e., in wet lab/on device) success of designed gRNA as well as for the implementation of the designed gRNA in gene editing techniques.

Hence, the Cas-CRISPR Automated Design and Evaluation (CasCADE) platform is a pioneering bioinformatics tool designed to streamline the CRISPR assay setup and offers the following advantages compared to traditional gRNA design processes: a) CasCADE uniquely facilitates the selection and optimization of essential parameters such as inclusion and exclusion groups, Cas protein types, scaffold sequences, Protospacer Adjacent Motif (PAM), and k-mer sizes; and b) CasCADE enhances the process of identifying conserved sequences across selected genomic datasets, assessing them for critical properties such as structural integrity, energy stability, and GC content.

These improvements over existing art not only enable the CasCADE platform to accelerate the development of robust gRNA sequences but also ensures the high specificity and inclusivity of the output gRNA designs, which can be essential for distinguishing target pathogens accurately. By providing rapid outputs and integrating a user-friendly approach for data handling, CasCADE can represent a significant innovation in the rapid deployment of CRISPR-based diagnostics, potentially reshaping the landscape of pathogen detection and public health monitoring.

It should be understood that the present disclosure is not limited to the details or methodology set forth in the description or illustrated in the figures. It should also be understood that the terminology used herein is for the purpose of description only and should not be regarded as limiting.

It is also to be understood that various aspects disclosed herein can be combined in different combinations than the combinations specifically presented in the description and accompanying drawings. It should also be understood that, depending on the example, certain acts or events of any of the processes or processes described herein can be performed in a different sequence, can be added, merged, or left out altogether (e.g., all described acts or events may not be necessary to conduct the techniques). In addition, although certain aspects of this disclosure can be described as being performed by a single module or unit for purposes of clarity, it should be understood that the techniques of this disclosure can be performed by a combination of units or modules.

In one or more instances, the present system can transform a plurality of data elements for analysis via training model processes and other techniques described herein. In certain instances, the present system can clean and transform data to remove, impute, or otherwise modify missing, null, or erroneous data values. In various instances, the present system can remove identifying information in order to anonymize and remove any correlated data. Similarly, the system can index and correlate specific data elements, data types, and data sets.

In one or more examples, the described techniques can be implemented in hardware, software, firmware, or any combination thereof.

Computer program code that implements the functionality described herein typically comprises one or more program modules that can be stored on a data storage device. This program code can include an operating system, one or more application programs, other program modules, and program data. The assay designer can enter commands and information into the computer through a keyboard, touch screen, pointing device, a script containing computer program code written in a scripting language, or other input devices, such as a microphone, etc. These and other input devices are often connected to a processing unit through known electrical, optical, or wireless connections.

The computer that affects many aspects of the described processes typically operates in a networked environment using logical connections to one or more remote computers or data sources. Remote computers can be another personal computer, a server, a router, a network PC, or other common network nodes, and typically include many or all of the elements described above relative to the main computer system in which the systems are embodied. The logical connections between computers include a LAN, a WAN, virtual networks (WAN or LAN), and wireless LAN (“WLAN”) that are presented here by way of example and not limitation. Such networking environments are commonplace in office-wide or enterprise-wide computer networks, intranets, and the Internet.

When used in a LAN or WLAN networking environment, a computer system implementing aspects of the system is connected to the local network through a network interface or adapter. When used in a WAN or WLAN networking environment, the computer can include a modem, a wireless link, or other mechanisms for establishing communications over the WAN, such as the Internet. In a networked environment, program modules depicted relative to the computer, or portions thereof can be stored in a remote data storage device. The network connections described or shown are non-limiting examples and other mechanisms of establishing communications over WAN or the Internet can be used.

Additional aspects, features, and processes of the claimed systems are readily discernible from the description herein, by those of ordinary skill in the art. Many embodiments and adaptations of the disclosure and claimed systems other than those herein described, as well as many variations, modifications, and equivalent arrangements and processes, are apparent from or reasonably suggested by the disclosure and the description thereof, without departing from the substance or scope of the claims. Furthermore, any sequence(s) and/or temporal order of steps of various processes described and claimed herein are those considered to be the best mode contemplated for conducting the claimed systems. It should also be understood that, although steps of various processes can be shown and described as being in a preferred sequence or temporal order, the steps of any such processes are not limited to being conducted in any particular sequence or order, absent a specific indication of such to achieve a particular intended result. In most cases, the steps of such processes can be conducted in a variety of different sequences and orders, while still falling within the scope of the claimed systems. In addition, some steps can be conducted simultaneously, contemporaneously, or in synchronization with other steps.

Aspects, features, and benefits of the claimed systems and processes for using the same are apparent from the information disclosed in the exhibits as incorporated by reference. Variations and modifications to the disclosed systems and processes can be affected without departing from the spirit and scope of the novel concepts of the disclosure. No limitation of the scope of the disclosure is intended by the information disclosed in the exhibits; any alterations and further modifications of the described or illustrated embodiments, and any further applications of the principles of the disclosure as illustrated therein are contemplated as would normally occur to one skilled in the art to which the disclosure relates. The description of the disclosed embodiments has been for illustration and description and is not intended to be exhaustive or to limit the devices and processes for using the same to the precise forms disclosed. Many modifications and variations are possible in light of the above teaching.

The embodiments were chosen and described to explain the principles of the devices and processes for using the same and their practical application to enable others skilled in the art to utilize the devices and processes for using the same. Various embodiments with various modifications are suited to the particular use contemplated. Alternative embodiments will become apparent to those skilled in the art to which the present devices and processes for using the same pertain without departing from their spirit and scope. Accordingly, the scope of the present devices and processes for using the same is defined by the appended claims rather than the description and the embodiments described therein.

CLWHAT is claimed is:

1. A process for designing gRNA sequences for use in CRISPR-based detection assays, the process comprising:
analyzing an input data set comprising one or more target inclusive sequences and a selected Cas protein;
identifying one or more conserved k-mers within the data set, wherein the one or more conserved k-mers are substantially equal to a size required by the selected Cas protein;
concatenating one or more scaffold sequences with the one or more identified conserved k-mers to create one or more candidate gRNA sequences;
evaluating structural and specificity characteristics of the one or more candidate gRNA sequences; and
displaying one or more output gRNA sequences, wherein the one or more output gRNA sequences are a subset of the one or more candidate gRNA sequences, the subset created by removing any candidate gRNA sequences of the one or more candidate gRNA sequences not abiding to the structural and specificity requirements.

2. The process of claim 1, further comprising retrieving one or more genome data sets associated with the one or more target inclusive sequences from a National Center for Biotechnology Information database.

3. The process of claim 1, further comprising creating a genome data set using metadata associated with the genome data set.

4. The process of claim 1, further comprising receiving requirements of the structural characteristics, the structural characteristics comprising at least one of a PAM sequence location, guanine/cytosine (GC) content, a scaffold sequence free energy, a gRNA free energy, and preservation of the scaffold sequence folding structure upon addition of the candidate gRNA sequence.

5. The process of claim 4, further comprising identifying a PAM region within the one or more target inclusive sequences in a location required by the selected Cas protein.

6. The process of claim 4, further comprising identifying a PAM region of a length required by the selected Cas protein within the one or more target inclusive sequences.

7. The process of claim 4, wherein GC content of the candidate gRNA sequences is required to be between 40% and 60%.

8. The process of claim 1, further comprising utilizing clustering or graphing operations to identify the one or more conserved k-mers among a set of k-mers selected for analysis.

9. The process of claim 1, wherein the selected Cas protein is Cas12.

10. The process of claim 1, wherein the selected Cas protein is Cas13.

11. The process of claim 1, wherein an inclusive group and an exclusive group are defined using a BLAST database,
wherein the inclusive group comprises a record of all genomes associated with the target inclusive sequences; and
wherein the exclusive group comprises a record of one taxonomic tree-level above a taxonomy of the inclusive group.

12. The process of claim 11, further comprising evaluating a specificity characteristic of inclusivity by determining matches between the one or more candidate gRNA sequences and the inclusive group and evaluating a specificity characteristic of exclusivity by determining matches between the candidate gRNA sequences and the exclusive group.

13. The process of claim 12, wherein at least one candidate gRNA sequence is at least 98% inclusive.

14. The process of claim 12, wherein at least one candidate gRNA sequence is at least 98% exclusive to taxonomic near neighbors.

15. The process of claim 11, further comprising evaluating a specificity characteristic of exclusivity to human signal by determining matches between the candidate gRNA sequences and the GRCh38 human genome.

16. The process of claim 15, wherein at least one candidate gRNA sequence is at least 98% exclusive to the human genome.

17. The process of claim 1, further comprising experimentally validating the output gRNA sequences via an experimental assay.

18. A system for designing gRNA sequences for use in CRISPR-based detection assays, the system comprising:
a computing device configured to receive at least one input data set comprising one or more target inclusive sequences and a selected Cas protein, the computing device comprising:
a processor; and
a memory device comprising a non-transitory storage medium encoded with instructions executable by the processor which, when executed by the processor, cause the processor to:
identify one or more conserved k-mers within the at least one input data set, wherein the one or more conserved k-mers are substantially equal to a size required by the selected Cas protein;
concatenate one or more scaffold sequences with the one or more identified conserved k-mers to create one or more candidate gRNA sequences;
evaluate structural and specificity characteristics of the one or more candidate gRNA sequences; and
display one or more output gRNA sequences, wherein the one or more output gRNA sequences are a subset of the one or more candidate gRNA sequences, the subset created by removing any candidate gRNA sequences of the one or more candidate gRNA sequences not abiding to the structural and specificity requirements.

19. The system of claim 18, wherein the instructions are coded in python script.

20. The system of claim 18, wherein the subset of output gRNA sequences is produced within 24 hours of receiving the input data set of target inclusive sequences and the selected Cas protein to the system.


SEQUENCE LISTING (34 sequences)

SEQ ID NO: 1 - RNA, 20 nt, synthetic construct
  source 1..20: mol_type = other RNA
cactgaacgtatagacccat

SEQ ID NO: 2 - RNA, 20 nt, synthetic construct
  source 1..20: mol_type = other RNA
tgatagcttgagatcccatg

SEQ ID NO: 3 - RNA, 20 nt, synthetic construct
  source 1..20: mol_type = other RNA
gtctctatggaatcactgcg

SEQ ID NO: 4 - RNA, 20 nt, synthetic construct
  source 1..20: mol_type = other RNA
atggccaatcctgcagaatc

SEQ ID NO: 5 - RNA, 20 nt, synthetic construct
  source 1..20: mol_type = other RNA
tgttctccatgagtccccct

SEQ ID NO: 6 - RNA, 20 nt, synthetic construct
  source 1..20: mol_type = other RNA
acttcacgttcggttcatcc

SEQ ID NO: 7 - RNA, 20 nt, synthetic construct
  source 1..20: mol_type = other RNA
tagagaatgacagacggctt

SEQ ID NO: 8 - RNA, 20 nt, synthetic construct
  source 1..20: mol_type = other RNA
cattttcaaagccgccggaa

SEQ ID NO: 9 - RNA, 20 nt, synthetic construct
  source 1..20: mol_type = other RNA
gccagaccgatgttaaacct

SEQ ID NO: 10 - RNA, 20 nt, synthetic construct
  source 1..20: mol_type = other RNA
atcgcatcaaactcttcggt

SEQ ID NO: 11 - RNA, 20 nt, synthetic construct
  source 1..20: mol_type = other RNA
atcaattggtcgcaggttcg

SEQ ID NO: 12 - RNA, 20 nt, synthetic construct
  source 1..20: mol_type = other RNA
gatacttcgaacgcatgacc

SEQ ID NO: 13 - RNA, 20 nt, synthetic construct
  source 1..20: mol_type = other RNA
tctatcgcggtgatttgtcg

SEQ ID NO: 14 - RNA, 20 nt, synthetic construct
  source 1..20: mol_type = other RNA
gacctacgatacgcactatg

SEQ ID NO: 15 - RNA, 20 nt, synthetic construct
  source 1..20: mol_type = other RNA
ttttggagctgtttactggc

SEQ ID NO: 16 - RNA, 41 nt, synthetic construct
  source 1..41: mol_type = other RNA
taatttctactaagtgtagatcactgaacgtatagacccat

SEQ ID NO: 17 - RNA, 41 nt, synthetic construct
  source 1..41: mol_type = other RNA
taatttctactaagtgtagattgatagcttgagatcccatg

SEQ ID NO: 18 - RNA, 41 nt, synthetic construct
  source 1..41: mol_type = other RNA
taatttctactaagtgtagatgtctctatggaatcactgcg

SEQ ID NO: 19 - RNA, 41 nt, synthetic construct
  source 1..41: mol_type = other RNA
taatttctactaagtgtagatatggccaatcctgcagaatc

SEQ ID NO: 20 - RNA, 41 nt, synthetic construct
  source 1..41: mol_type = other RNA
taatttctactaagtgtagattgttctccatgagtccccct

SEQ ID NO: 21 - RNA, 41 nt, synthetic construct
  source 1..41: mol_type = other RNA
taatttctactaagtgtagatacttcacgttcggttcatcc

SEQ ID NO: 22 - RNA, 41 nt, synthetic construct
  source 1..41: mol_type = other RNA
taatttctactaagtgtagattagagaatgacagacggctt

SEQ ID NO: 23 - RNA, 41 nt, synthetic construct
  source 1..41: mol_type = other RNA
taatttctactaagtgtagatcattttcaaagccgccggaa

SEQ ID NO: 24 - RNA, 41 nt, synthetic construct
  source 1..41: mol_type = other RNA
taatttctactaagtgtagatgccagaccgatgttaaacct

SEQ ID NO: 25 - RNA, 41 nt, synthetic construct
  source 1..41: mol_type = other RNA
taatttctactaagtgtagatatcgcatcaaactcttcggt

SEQ ID NO: 26 - RNA, 41 nt, synthetic construct
  source 1..41: mol_type = other RNA
taatttctactaagtgtagatatcaattggtcgcaggttcg

SEQ ID NO: 27 - RNA, 41 nt, synthetic construct
  source 1..41: mol_type = other RNA
taatttctactaagtgtagatgatacttcgaacgcatgacc

SEQ ID NO: 28 - RNA, 41 nt, synthetic construct
  source 1..41: mol_type = other RNA
taatttctactaagtgtagattctatcgcggtgatttgtcg

SEQ ID NO: 29 - RNA, 41 nt, synthetic construct
  source 1..41: mol_type = other RNA
taatttctactaagtgtagatgacctacgatacgcactatg

SEQ ID NO: 30 - RNA, 41 nt, synthetic construct
  source 1..41: mol_type = other RNA
taatttctactaagtgtagatttttggagctgtttactggc

SEQ ID NO: 31 - RNA, 39 nt, synthetic construct
  source 1..39: mol_type = other RNA
aatttctactgttgtagatgtcgtgctaaactttactag

SEQ ID NO: 32 - RNA, 39 nt, synthetic construct
  source 1..39: mol_type = other RNA
gatcatttcaaatcgtgctgtagatgttgtcatctttaa

SEQ ID NO: 33 - RNA, 39 nt, synthetic construct
  source 1..39: mol_type = other RNA
aatttctactgttgtagatcagtgggttcggtcctccag

SEQ ID NO: 34 - RNA, 39 nt, synthetic construct
  source 1..39: mol_type = other RNA
gacctcctggcttgggtgactagatgttgtcatctttaa